(19) United States
(12) Patent Application Publication
(10) Pub. No.: US 2004/0116501 A1
(43) Pub. Date: Jun. 17, 2004

(54) Treatment of nonallergic rhinitis by selective phosphodiesterase 4 inhibitors

(76) Inventors: Chris Rundfeldt, Coswig (DE); Hildegard Kuss, Dresden (DE); Norbert Hofgen, Medingen (DE)

Correspondence Address: FULBRIGHT & JAWORSKI, LLP, 666 FIFTH AVE, NEW YORK, NY, 10103-3198, US

(21) Appl. No.: 10/654,365

(22) Filed: Sep. 3, 2003

(30) Foreign Application Priority Data
Sep. 6, 2002 (DE) 102 41 407.6

(51) Int. Cl. A61K 31/405

(52) U.S. Cl. 514/411; 514/414

(57) ABSTRACT

The invention relates to the use of hydroxyindolylglyoxylamides as inhibitors of phosphodiesterase 4 for the treatment of nonallergic rhinitis.

The invention relates to the use of hydroxyindolylglyoxylamides as inhibitors of phosphodiesterase 4 for the treatment of nonallergic rhinitis.

A whole series of disorders which involve the symptoms of chronic rhinitis but which do not have an allergic origin are referred to as nonallergic rhinitis. The general symptoms occurring with nonallergic rhinitis are nasal blockage/congestion and nasal discharge without the symptoms of sneezing and conjunctival irritation. Frequent sneezing and irritation of the conjunctiva are symptoms appearing mainly in association with allergic rhinitis. Patients with nonallergic rhinitis have negative or clinically irrelevant allergic skin tests and a normal serum IgE level. Although the existence of nonallergic rhinitis is difficult to determine, the figures published by the National Rhinitis Classification Task Force in the USA were that 23% of all patients suffering from chronic rhinitis have nonallergic rhinitis, 34% have mixed forms and 43% have allergic rhinitis.

Included among persistent disorders of unknown origin is vasomotor rhinitis, a chronic iodiopathic disease which is not infectious, shows no elevated serum IgE levels and is not associated with an inflammation and/or eosinophilia. It is the commonest form of nonallergic rhinitis with the main symptoms of congestion and nasal discharge. The pathophysiology of this disorder is unclear, and the nasal hyperreactivity which occurs is triggered by nonimmunological stimuli such as cold air, cigarette smoke, chemical irritants, strong odour or physical exertion and stress (Zeiger, R. S., Allergic and Nonallergic Rhinitis. Classification and Pathogenesis: Part II. Nonallergic rhinitis, American Journal of Rhinology (1989), 3:113-139).

The nonallergic rhinitis with eosinophils syndrome (NARES) is characterized by episodic sneezing, watery nasal discharge, nasal irritation and eosinophilia in the nasal smears without signs of an allergy. The symptoms occurring in NARES are more intense than in vasomotor rhinitis or allergic rhinitis. NARES is an isolated disease which occurs as accompaniment to asthma, aspirin hypersensitivity or in association with nasal polyps.

The etiology of some other rhinitis syndromes is somewhat clearer. Chronic sinusitis is a chronic mucosal inflammation not caused by bacteria in most cases. Rhinitis medicamentosa is caused by a number of substances such as beta-blockers, ACE inhibitors, oral contraceptives or prazosin. It is characterized more by interstitial edema than by vasodilatation. Nasal polyps are commonly associated with chronic nonallergic rhinitis and make its symptoms more pronounced (Zeiger (1989), supra).

Besides the rhinitis syndromes mentioned, nonallergic rhinitis very often occurs as a symptom of infections of the airways including the paranasal sinuses. The infections in these cases are caused by viruses, bacteria, fungi or by combinations of the microbes mentioned. Protozoa or multicellular parasites are a less common cause of rhinitides. The infection, i.e. the colonization by the microbes mentioned, may in these cases, such as, for example, in an infection with rhinoviruses, be restricted locally to the mucosa of the upper airways or, as in the case of influenza virus infection, affect the whole body in addition to the upper airways. It is common to all these infections that an inflammatory change of the nasal mucosa is to be observed as symptom. This may have a more exudative character (running nose) or a more edematous character (nose blocked by swelling). The majority of these infectious diseases have an acute course, but chronic courses are known. These chronic rhinitides often lead, as do other chronic (noninfectious) rhinitides also, to proliferations of the nasal mucosa, the so-called nasal polyps.

Allergic rhinitis, also referred to as hay fever, differs distinctly from the types of nonallergic rhinitis. Allergic rhinitis is based, as are all allergic disorders, on a chronic, continually progressive complex cellular inflammatory response characterized by an increased accumulation of eosinophilic granulocytes and an elevated serum IgE level. Allergic rhinitis is induced by a hypersensitivity to allergens such as pollen, house dust, mites, animal hair or chemical substances.

The main symptoms of allergic rhinitis are increased nasal discharge, nasal congestion due to formation of edema, frequent sneezing and irritation of the conjunctiva. The principal aim of therapy besides treatment of the symptoms is to suppress the inflammation, which is controlled by mediators, of the nasal mucosa. Despite intensive research activity, the pathogenesis of the allergic disorders has not been completely elucidated yet.

Although a large number of medicaments can currently be employed for the therapy of various types of rhinitis, the treatment is unsatisfactory in many cases. The treatment of the types of nonallergic rhinitis in particular is inadequate, and these disorders often display a chronic course despite the use of drug products.

The therapeutic agents currently regarded as most effective for nonallergic and allergic rhinitis are topically or orally administered corticosteroids (steroids). Steroids are often associated with serious side effects on prolonged use (such as osteoporosis, growth retardation) (Forth, W., Henschler, D., Rummel, W., Starke, K., Allgemeine und spezielle Pharmakologie und Toxikologie, Bibliographisches Institut & F. A. Brockhaus A G, Mannheim (1993), 562-563). Accordingly, they are often employed by the patients and by the treating physicians only in the advanced phase of the disorder. The risks associated with this are that 1.) bronchial asthma will develop from a relatively mild rhinitis (rhinoconjunctivitis) (stage shift) and 2.) the inflammation underlying the disorder will progress. There will consequently be remodeling of the tissue structure of the lower airways. In place of the reversal changes there are irreversible morphological remodeling processes which lead to narrowing of the airways. Further drug products employed in the symptomatic treatment of nonallergic rhinitis are topical antihistamines, anticholinergics or vasoconstrictors, which inhibit nasal discharge but have no effect on the tissue inflammation and give no relief when there is nasal obstruction. Some of these agents may be used only short-term, because on prolonged use the tissue is destroyed (vasoconstrictors e.g. alpha-adrenergic substances (Bachert, C., Ganzer, U., Die nasale Hyperreaktivität. Die allergische Rhinitis und ihre Differentialdiagnosen—Konsensusbericht zur Pathophysiologie, Klassifikation, Diagnose und Therapie.; Nasal hyperractivity. Allergic rhinitis and differential diagnoses—consensus report on pathophysiology, classification, diagnosis and therapy Laryngo-rhino-otologie (1997), 72(2) 65-76).

It is known that selective inhibitors of the PDE4 isoenzyme can be employed for the therapy of allergic rhinitis or of allergic bronchial asthma. These isoenzymes have various functions in the body and are expressed differently in the individual cell types (Beavo, J. A., Conti, M. and Heaslip, R. J., Multiple cyclic nucleotide phosphodiesterases. Mol. Pharmacol, 1994, 46:399-405; Hall I P., Isoenzyme selective phosphodiesterase inhibitors: potential clinical uses, Br. J. clin. Pharmacol. 1993, 35:1-7). Inhibition of the various types of PDE isoenzymes results in accumulation of cAMP or cGMP in the cells, which can be utilized for therapy (Torphy, T. J., Livi, G. P., Christensen, S. B., Novel Phosphodiesterase Inhibitors for the Therapy of Asthma, Drug News and Perspectives 1993, 6:203-214; Torphy, T. J., Phosphodiesterase isoenzymes: Molecular targets for novel antiasthmatic agents. Am J Respir Crit Care Med 1998; 157:351-370).

Cyclic adenosine monophosphate (cAMP) is one of the so-called intracellular messengers whose intracellular concentration is regulated by the phosphodiesterase (PDE) isoenzymes. Studies have revealed that selective inhibitors of the PDE4 isoenzyme raise the intracellular concentration of cAMP and thus inhibit the proinflammatory activity of a large number of cells (e.g. eosinophilic and neutrophilic granulocytes). Selective PDE4 inhibitors also inhibit the release of histamine from the mast cells or stabilize the endothelial cells of the blood vessels in the nasal mucosa, making these active substances also suitable for treating the acute symptoms of allergic rhinoconjunctivitis (Barnette, M. S., Phosphodiesterase 4 (PDE4) inhibitors in asthma and chronic obstructive pulmonary disease (COPD). Progress Drug Research (1999), 53, E. Jucker Ed., Birkhäuser Verlag, Basle (Switzerland); Dyke, H. J. and Montana, J. G., The therapeutic potential of PDE4 inhibitors, Exp Opin Invest Drugs, (1999), 8(9):1301-1325).

DE 198 18 964 A1 describes hydroxyindolylglyoxylamides as PDE4 inhibitors. A particularly preferred compound is the compound N-(3,5-dichloropyrid-4-yl)-[1-(4-fluorobenzyl)-5-hydroxyindol-3-yl]glyoxylamide (AWD 12-281). These hydroxyindolylglyoxylamides can be employed for treating inflammatory airway disorders such as allergic rhinitis. Further preferred compounds include AWD 12-322 (N-(3,5-dichloropyrid-4-yl)-2-[5-hydroxy-1-(4-hydroxybenzyl)-1H-indol-3-yl]glyoxylamide) and AWD 12-298 (N-(3,5-dichloropyrid-4-yl)-[1-(2,6-difluorobenzyl)-5-hydroxyindol-3-yl]glyoxylamide semi-ethyl acetate).

However, the effect of PDE4 inhibitors on the types of nonallergic rhinitis is completely unexpected and novel. The effect of the selective PDE4 inhibitors in these types of disorders of the nasal mucosa and of the bronchial epithelium of the upper respiratory tract has not previously been described. Another unknown fact is that PDE4 inhibitors are able to prevent the toxic effect of chemical substances such as acetic acid on tissue and, in particular, mucosa. It was possible to show in animal experiments with a model of the symptoms of vasomotor nonallergic rhinitis that PDE4 inhibitors have an excellent effect compared with standard therapeutic agents for the vasomotor rhinitis. Standard medicaments for this type of the disorder are steroids such as, for example, beclomethasone or anticholinergics such as ipratropium bromide. These substances show a dose-dependent effect against the vascular plasma permeability induced by acetic acid in the nasal mucosa in the rat rhinitis model.

Vasomotor rhinitis is one of the most commonly occurring forms of nonallergic rhinitis. Excessive watery rhinorrhea is induced in patients by parasympathetic hyperreactivity through stimulation or toxic irritation of the parasympathetic nerves of the nasal mucosa. Substances which greatly dilate the blood vessels are likewise able to induce heavy nasal discharge and formation of mucosal edema.

This nonallergic form of increased nasal discharge can be induced in animal experiments by the action of acetic acid on the nasal mucosa of the animals. The occurrence of an increased watery rhinitis after exposures to acetic acid is induced by two causes. Inhaled acetic acid vapor or superfusion of the nasal mucosa with acetic acid on the one hand induces an immediate dilatation of the blood vessels of the nasal mucosa, leading to high vascular permeability. Since this effect can be inhibited by the sensory neurotoxin capsaicin, the toxic effect of acetic acid is attributed to the irritation of sensory nerve fibers in the nasal mucosa (Stanek, J., Symanowicz, P. T., Olsen, J. E., Gianutsos, G., Morris, J. B., Sensory-nerve mediated nasal vasodilatory response to inspired acetaldehyde and acetic acid vapors, Inhalation toxicology (2001), 13(9):807-822). On the other hand, acetic acid is able to increase parasympathetic activity of the sensory nerve fibers in the nasal mucosa. Chemical substances with a strong odor like acetic acid also cause neural reflex stimulation of the parasympathetic activity of the nasal glands, resulting in overproduction of watery secretion and thus an increased nasal discharge. The effect of acetic acid on the mucosa is toxic and leads to a loss or adenosine triphosphate (ATP) in the tissue cells. The loss of ATP in the cells of the smooth muscles and the endothelial cells in the blood vessels leads to laxness of the cells and thus dose-dependently to vasodilatation (Kilgour, J. D., Simpson, S. A., Alexander, D. J., Reed, C. J., A rat nasal epithelial model for predicting upper respiratory tract toxicity in vivo-in vitro correlations, Toxicology (2000), 145(1):39-49).

One aspect of the invention relates to the use of hydroxyindol-3-ylglyoxylamides of the formula (I)

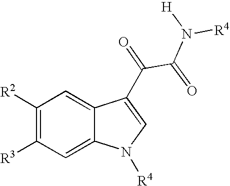

in which

R¹ is —C₁-C₆-alkyl, straight-chain or branched-chain, saturated or partially unsaturated, where appropriate substituted one or more times by mono-, bi- or tricyclic saturated or mono- or polyunsaturated carbocycles having 3-14 ring members or mono-, bi- or tricyclic saturated or mono- or polyunsaturated heterocycles having 5-15 ring members and 1-6 heteroatoms, which are preferably N, O and S,

where the carbocyclic and heterocyclic substituents in turn may be substituted where appropriate one or more times by —OH, —SH, —NH₂, —NHC₁-C₆-alkyl, —N(C₁-C₆-alkyl)₂, —NHC₆-C₁₄-aryl, —N (C₆-C₁₄-aryl)₂, —N(C₁-C₆-alkyl)(C₆-C₁₄-aryl), —NO₂, —CN, —F, —Cl, —Br, —I, —O—C₁-C₆-alkyl, —O—C₆-C₁₄-aryl, —C₁-C₆-alkyl, —C₆-C₁₄-aryl or/and —COOH,

where each C₁-C₆-alkyl radical on the carbocyclic and heterocyclic substituents may itself be substituted one or more times by —F, —Cl, —Br, —I, —OH or/and C₆-C₁₄-aryl, and where each C₆-C₁₄-aryl radical on the carbocyclic and heterocyclic substituents may itself be substituted one or more times by —F, —Cl, —Br, —I, —OH or/and C₁-C₆-alkyl,

R², R³ may be hydrogen or —OH, it being necessary for at least one of the two substituents to be —OH;

R⁴ is a mono- or polycyclic aromatic carbocycle having 6-14 ring members or a mono- or polycyclic heterocycle having 5-15 ring members, where the heteroatoms are selected from N, O and S,

where appropriate substituted one or more times by —F, —Cl, —Br, —I, —OH, —SH, —NH₂, —NH(C₁-C₆-alkyl), —N(C₁-C₆-alkyl)₂, —NH (C₆-C₁₄-aryl), —N (C₆-C₁₄-aryl)₂, —N(C₁-C₆-alkyl)(C₆-C₁₄-aryl), —NO₂, —CN, —O—C₁-C₆-alkyl, —O—C₆-C₁₄-aryl, —C₁-C₆-alkyl, —C₆-C₁₄-aryl or/and —COOH,

where each C₁-C₆-alkyl radical may itself be substituted one or more times by —F, —Cl, —Br, —I, —OH or/and —C₆-C₁₄-aryl and each C₆-C₁₄-aryl radical may itself be substituted one or more times by —F, —Cl, —Br, —I, —OH or/and C₁-C₆-alkyl, for the treatment of nonallergic rhinitis.

R¹ is preferably a C₁-C₃-alkyl radical which is substituted where appropriate, such as, for example, n-propyl, isopropyl, cyclopentylmethyl or a benzyl radical which may itself be substituted one or more times by halogen, e.g. —F, —O—C₁-C₆-alkyl or —O—C₁-C₆-haloalkyl, e.g. —OCH₃ or OCF₃, or/and —C₁-C₆-alkyl or C₁-C₆-haloalkyl, e.g. —CH₃ or —CF₃.

R⁴ is preferably mono- or bicyclic aromatic carbocycles or heterocycles. R⁴ is particularly preferably phenyl or pyridyl, in particular 4-pyridyl.

It is further preferred for R⁴ to be substituted one or more times by —F, —Cl, —Br or/and —I. The most preferred compound is AWD 12-281.

Further preferred compounds include AWD 12-322 (N-(3,5-dichloropyrid-4-yl)-2-[5-hydroxy-1-(4-hydroxybenzyl)-1H-indol-3-yl]glyoxylamide) and AWD 12-298 (N-(3,5-dichloropyridin-4-yl)-[1-(2,6-difluorobenzyl)-5-hydroxyindol-3-yl]glyoxylamide semi-ethyl acetate).

Besides the compounds of the formula (I), it is also possible to employ pharmacologically acceptable salts thereof. The pharmacologically acceptable salts can be obtained by neutralizing the compounds with suitable organic or inorganic bases or acids.

Compounds of the formula (I) can be employed for the therapeutic treatment or/and for the prevention of various types of nonallergic rhinitis, e.g. vasomotor rhinitis, nonallergic rhinitis with eosinophilia syndrome, chronic sinusitis, rhinitis medicamentosa and other types of nonallergic rhinitis.

The compounds of the invention are preferably administered in the form of pharmaceutical compositions which, besides the active ingredient, comprise pharmacologically acceptable carriers, excipients or diluents.

The dosage of the active ingredients may vary depending on the route of administration, age, weight of the patient, nature and severity of the disorders to be treated and similar factors. The daily dose can be given as a single dose to be administered once a day or divided into two or more daily doses, and is ordinarily from 0.001 to 100 mg, e.g. 0.01 to 50 mg.

Examples of suitable administration forms are oral, parenteral, intravenous, transdermal, topical, inhalational and intranasal preparations, with preference for inhalational and intranasal preparations.

The conventional pharmaceutical formulations are used, such as tablets, coated tablets, capsules, dispersible powders, granules, aqueous solutions, aqueous or oily suspensions, syrup, liquids or drops. Administration particularly preferably takes place in the form of atomized liquid preparations, e.g. in the form of aerosols or sprays.

The compounds of the invention or the pharmaceutical products comprising these compounds can also be administered in combination with other pharmacological active ingredients such as, for example, products having anti-inflammatory activity, e.g. corticosteroids (steroids) (e.g. beclomethasone) or leukotriene antagonists (e.g. montelukast), secretion inhibitors such as, for example, anticholinergics (e.g. ipratropium bromide), antihistamines such as, for example, azelastine, vasoconstrictors such as, for example, xylometazoline hydrochloride, medicaments having antiviral activity, such as, for example, oseltamivir or adamantane, products having antibacterial activity, such as antibiotics (e.g. penicillin) or products having antifungal (fungicidal or fungistatic) effects.

The invention is also to be illustrated by the following example.

### EXAMPLE

### Effect on Acetic Acid-Induced Vascular Permeability in Sprague-Dawley Rats (Nonallergic Rhinitis Model)

Male Sprague-Dawley rats weighing 280-320 g are anesthetized on the day of the experiment by i.p. injection of 0.9-1 ml/animal of a 2.5% strength thiopental sodium solution. The trachea is then exposed below the epiglottis and incised at two points. A polyethylene catheter is pushed into the lower opening in the trachea in the direction of the lung (orthograde) and secured by tying with twine in order to maintain breathing. A second polyethylene catheter with LUER lock connector (cut from Original-Perfusor® line) for retrograde perfusion of the nasal cavity is introduced into the upper opening and advanced retrogradely as far as the inner opening of the choanae so that the solution can flow through the nasal cavity. 8 animals in total are placed individually on their backs on specially fabricated plastic tables in such a way that the perfusion fluid can drip out of the nostrils and be collected in the fraction collector.

During this, half of the head looks with the nose beyond the edge of the table.

A polyethylene infusion line is connected to the LUER lock connector of the catheter which has been secured retrogradely in each animal and is immersed in the container with the prepared perfusion fluid via a roller pump. The roller pump is adjusted to a constant delivery of 0.5 ml of fluid/min. A red lamp is switched on over the animals for warming.

Initial perfusion of the nasal cavity with PBS for 30 min serves to wash out the nasal mucus. During this, the fraction collector is switched on and the perfusate dripping out of the nostrils is collected in 15 min fractions (2 fractions). The second fraction is used as normal value for the experiment.

The test substances are administered topically before administration of acetic acid. They are added to the perfusion medium (PBS, Dulbecco) in molar concentrations. 4.6 mg of AWD 12-281 or other test substances such as AWD 12-322 or AWD 12-298 are diluted in accordance with their molecular weight (4.6 mg of AWD 12-322 or 5.2 mg of AWD 12-298) with 1 ml of 1N NaOH and then made up to 10 ml with double-distilled H₂O. 5.2 mg of beclomethasone are treated with 2 ml of 1N NaOH and 4 ml of 96% pure ethanol in an ultrasonic bath for 2 min and then made up to 10 ml with double-distilled H₂O, and PBS is added to give the final concentration, as reference 1. 1.4 mg of ipratropium bromide are diluted with 1 ml of 1N NaOH and then made up to 10 ml with double-distilled H₂O, and PBS is added to give the final concentration, as reference 2. These solutions are perfused through the noses using the roller pump for 30 min, and the 2 samples obtained from the fraction collector are discarded. The roller pump is then switched off.

After perfusion with the substance, the plasma marker Evans Blue (1% strength solution in PBS) is injected, 1 ml per animal, into the jugular vein and then 0.1% strength acetic acid solution is forced through tubings and roller pumps into the nasal cavity until 2-3 drops of acetic acid solution drip from the conchae. The roller pumps are then switched off. The acetic acid solution is left in the nasal cavity for 30 minutes in order to ensure complete permeation of the nasal mucosa.

After an exposure time of 30 minutes, the roller pumps and the fraction collector are switched on again, and the 0.1% strength acetic acid solution in PBS is perfused retrogradely (with a constant delivery of 0.5 ml liquid/min) through the nasal cavity for 60 min. During this, the perfusate dripping out of the nostrils is continuously received in glass tubes and collected in 4 periods of 15 min. Aliquots of the samples including the control samples are placed on microtiter plates and measured using a Digiscan photometer at a wavelength of 620 nm relative to the control sample (blank). The course of the effect on the vascular permeability of the nasal mucosa is calculated as AUC over 60 min (area under the curve, AUC_{0-60 min} in μg/l) (Grunwald, C., AUC 1.0, calculation and graphical representation of area under curve (AUC) values, internal report of Arzneimittelwerke Dresden GmbH, August 1995). The Evans Blue content in 4 collected fractions, each selected over 15 minutes, of the perfusate flowing out in μg/ml is calculated by formula 1:

$${AUC}_{0 \text{-} 60 min} = {\sum }_{n = 1}^{4} \{ Δ t \cdot \frac{{y}_{n - 1} + {y}_{n}}{2} \}$$

tₙ time interval (15 min)

yₙ Evans Blue content per time interval (μg/l per 15 min)

In order to calculate the acetic acid-induced vascular permeability over the course of one hour, the baseline value (2nd fraction of the initial perfusion with Dulbecco PBS) is subtracted):

AUC′_{0-60 min}=AUC_{0-60 min}−baseline value×60 min

The means and standard deviations (x±SD) of the AUC′ for individual animals in a control group or a group of animals pretreated with substance are calculated. The inhibition of the vascular permeability is stated in percent. The calculation takes place by setting the mean dye content of the fractions collected from control animals treated with vehicle at 100% and by comparison relating this to the mean dye content in fractions collected from animals treated prophylactically with substance (x% inhibition). Each substance concentration and each corresponding vehicle solution is tested on 4 to 8 animals.

Statistical Analysis:

Student's t test for unpaired observation is used to calculate the statistical significance. Values of p<0.05 and below are regarded as significant.

TABLE 1
Effect of AWD 12-281 on the acetic acid-
induced vascular permeability of the rat
nasal mucosa through a single 30-minute
perfusion (the substance was dissolved in the
perfusion fluid), 60 min before perfusion of
0.1% strength acetic acid solution

| | | | Evans Blue content | |
|---|---|---|---|---|
| | Test | | (μg/ml) in the | |
| | substance | | perfusate | |
| | concentration | | AUC′ | % |
| Test substance | [μmol/l] | n | x ± SD | Inhibition |
| Vehicle control | 0 | 8 | 15.29 ± 7.47 | 0 |
| AWD 12-281 | 0.1 | 8 | 10.88 ± 8.45 | 29 |
| Vehicle control | 0 | 8 | 15.30 ± 8.56 | 0 |
| AWD 12-281 | 1 | 8 | 5.96 ± 7.97 | 61* |
| Vehicle control | 0 | 8 | 15.29 ± 7.47 | 0 |
| AWD 12-281 | 3 | 8 | 6.41 ± 5.81 | 58* |
| Vehicle control | 0 | 8 | 15.29 ± 7.47 | 0 |
| AWD 12-281 | 10 | 8 | 3.68 ± 4.40 | 76** |

AUC′ = value of area under the curve taking account of the baseline value
n = number of animals per group
*, **p < 0.05, p < 0.01 statistical significance calculated by Student's t test comparing with the vehicle control group.

TABLE 2
Effect of AWD 12-322 on the acetic acid-
induced vascular permeability of the rat
nasal mucosa through a single 30-minute
perfusion (the substance was dissolved in the
perfusion fluid), 60 min before perfusion of
0.1% strength acetic acid solution

| | | | Evans Blue content | |
|---|---|---|---|---|
| | Test | | (μg/ml) in the | |
| | substance | | perfusate | |
| | concentration | | AUC′ | % |
| Test substance | [μmol/l] | n | \overline{x} ± SD | Inhibition |
| Vehicle control | 0 | 4 | 14.56 ± 4.68 | 0 |
| AWD 12-322 | 0.1 | 4 | 13.77 ± 6.65 | 5 |
| Vehicle control | 0 | 4 | 14.56 ± 4.68 | 0 |
| AWD 12-322 | 1 | 4 | 7.25 ± 6.77 | 50 |
| Vehicle control | 0 | 4 | 14.65 ± 4.68 | 0 |
| AWD 12-322 | 10 | 4 | 2.62 ± 3.44 | 82** |

AUC′ = value of area under the curve taking account of the baseline value
n = number of animals per group
**p < 0.01 statistical significance calculated by Student's t test comparing with the vehicle control group.

TABLE 3
Effect of AWD 12-298 on the acetic acid-
induced vascular permeability of the rat
nasal mucosa through a single 30-minute
perfusion (the substance was dissolved in the
perfusion fluid), 60 min before perfusion of
0.1% strength acetic acid solution

| | | | Evans Blue content | |
|---|---|---|---|---|
| | Test | | (μg/ml) in the | |
| | substance | | perfusate | |
| | concentration | | AUC′ | % |
| Test substance | [μmol/l] | n | \overline{x} ± SD | Inhibition |
| Vehicle control | 0 | 12 | 16.52 ± 11.77 | 0 |
| AWD 12-298 | 1 | 4 | 17.72 ± 9.31 | 0 |
| Vehicle control | 0 | 12 | 16.52 ± 11.77 | 0 |
| AWD 12-298 | 3 | 6 | 8.43 ± 5.56 | 49 |
| Vehicle control | 0 | 12 | 16.52 ± 11.77 | 0 |
| AWD 12-298 | 10 | 7 | 6.03 ± 7.11 | 64* |

AUC′ = value of area under the curve taking account of the baseline value
n = number of animals per group
*p < 0.05 statistical significance calculated by Student's t test comparing with the vehicle control group.

TABLE 4
Effect of beclomethasone on the acetic acid-
induced vascular permeability of the rat
nasal mucosa through a single 30-minute
perfusion (the substance was dissolved in the
perfusion fluid), 60 min before perfusion of
0.1% strength acetic acid solution

| | | | Evans Blue content | |
|---|---|---|---|---|
| | Test | | (μg/ml) in the | |
| | substance | | perfusate | |
| | concentration | | AUC′ | % |
| Test substance | [μmol/l] | n | x ± SD | Inhibition |
| Vehicle control | 0 | 4 | 17.37 ± 6.85 | 0 |
| Beclomethasone | 0.01 | 4 | 10.70 ± 2.38 | 38 |
| Vehicle control | 0 | 4 | 17.30 ± 6.85 | 0 |
| Beclomethasone | 0.1 | 4 | 4.93 ± 3.19 | 72* |
| Vehicle control | 0 | 4 | 17.37 ± 6.85 | 0 |
| Beclomethasone | 1 | 4 | 0.93 ± 1.80 | 95** |
| Vehicle control | 0 | 4 | 17.37 ± 6.85 | 0 |
| Beclomethasone | 3 | 4 | 5.51 ± 7.12 | 68* |
| Vehicle control | 0 | 4 | 25.04 ± 5.06 | 0 |
| Beclomethasone | 10 | 4 | 4.86 ± 8.08 | 81** |

AUC′ = value of area under the curve taking account of the baseline value
n = number of animals per group
*, **p < 0.05, p < 0.01 statistical significance calculated by Student's t test comparing with the vehicle control group.

TABLE 5
Effect of ipratropium bromide on the acetic
acid-induced vascular permeability of the rat
nasal mucosa through a single 30-minute
perfusion (the substance was dissolved in the
perfusion fluid), 60 min before perfusion of
0.1% strength acetic acid solution

| | | | Evans Blue content | |
|---|---|---|---|---|
| | Test | | (μg/ml) in the | |
| | substance | | perfusate | |
| | concentration | | AUC′ | % |
| Test substance | [μmol/l] | n | x ± SD | Inhibition |
| Vehicle control | 0 | 4 | 14.56 ± 4.68 | 0 |
| Ipratropium | 0.1 | 4 | 9.11 ± 10.31 | 37 |
| bromide | | | | |
| Vehicle control | 0 | 4 | 14.56 ± 4.68 | 0 |
| Ipratropium | 1 | 4 | 4.38 ± 7.55 | 70* |
| bromide | | | | |
| Vehicle control | 0 | 4 | 14.56 ± 4.68 | 0 |
| Ipratropium | 10 | 4 | 3.23 ± 5.70 | 78* |
| bromide | | | | |

AUC′ = value of area under the curve taking account of the baseline value
n = number of animals per group
*p < 0.05 statistical significance calculated by Student's t test comparing with the vehicle control group.

Results and Discussion:

The selective PDE4 inhibitors AWD 12-281 and 12-322 show in the test range from 0.1 to 10 μmol/l a concentration-dependent inhibition of the vascular permeability of the nasal mucosa in the model of acetic acid-induced rhinitis in rats. The derivative AWD 12-298 has concentration-dependent activity in the concentration range from 3 to 10 μmol/l. Compared with this, the standard therapeutic agents for the treatment of nonallergic rhinitis, such as the corticosteroid beclomethasone and the anticholinergic ipratropium bromide, have approximately the same activity. The inhibition by PDE4 inhibitors of the plasma extravasation induced by acetic acid is a completely unexpected and novel finding which has not previously been described.

1. The use of compounds of the general formula (I) 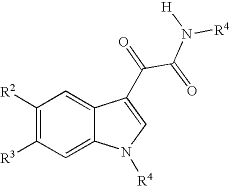
in which
R¹ is —C₁-C₆-alkyl, straight-chain or branched-chain, saturated or partially unsaturated, where appropriate substituted one or more times by mono-, bi- or tricyclic saturated or mono- or polyunsaturated carbocycles having 3-14 ring members or mono-, bi- or tricyclic saturated or mono- or polyunsaturated heterocycles having 5-15 ring members and 1-6 heteroatoms, which are preferably N, O and S,
where the carbocyclic and heterocyclic substituents in turn may be substituted where appropriate one or more times by —OH, —SH, —NH₂, —NHC₁-C₆-alkyl, —N(C₁-C₆-alkyl)₂, —NHC₆-C₁₄-aryl, —N(C₆-C₁₄-aryl)₂, —N (C₁-C₆-alkyl)(C₆-C₁₄-aryl), —NO₂, —CN, —F, —Cl, —Br, —I, —O—C₁-C₆-alkyl, —O—C₆-C₁₄-aryl, —C₁-C₆-alkyl, —C₆-C₁₄-aryl or/and —COOH,
where each C₁-C₆-alkyl radical on the carbocyclic and heterocyclic substituents may itself be substituted one or more times by —F, —Cl, —Br, —I, —OH or/and C₆-C₁₄-aryl, and where each C₆-C₁₄-aryl radical on the carbocyclic and heterocyclic substituents may itself be substituted one or more times by —F, —Cl, —Br, —I, —OH or/and C₁-C₆-alkyl,
R², R³ may be hydrogen or —OH, it being necessary for at least one of the two substituents to be —OH;
R⁴ is a mono- or polycyclic aromatic carbocycle having 6-14 ring members or a mono- or polycyclic heterocycle having 5-15 ring members, where the heteroatoms are selected from N, O and S,
where appropriate substituted one or more times by —F, —Cl, —Br, —I, —OH, —SH, —NH₂, —NH(C₁-C₆-alkyl), —N(C₁-C₆-alkyl)₂, —NH (C₆-C₁₄-aryl), —N(C₆-C₁₄-aryl)₂, —N (C₁-C₆-alkyl)(C₆-C₁₄-aryl), —NO₂, —CN, —O—C₁-C₆-alkyl, —O—C₆-C₁₄-aryl, —C₁-C₆-alkyl, —C₆-C₁₄-aryl or/and —COOH,
where each C₁-C₆-alkyl radical may itself be substituted one or more times by —F, —Cl, —Br, —I, —OH or/and —C₆-C₁₄-aryl and each C₆-C₁₄-aryl radical may itself be substituted one or more times by —F, —Cl, —Br, —I, —OH or/and C₁-C₆-alkyl,
for the treatment of nonallergic rhinitis.

2. The use as claimed in claim 1, characterized in that the compounds are N-(3,5-dichloropyrid-4-yl)-[1-(4-fluorobenzyl)-5-hydroxyindol-3-yl]glyoxylamide (AWD 12-281), N-(3,5-dichloropyrid-4-yl)-2-[5-hydroxy-1-(4-hydroxybenzyl)-1H-indol-3-yl]glyoxylamide (AWD 12-322) and N-(3,5-dichloropyrid-4-yl)-[1-(2,6-difluorobenzyl)-5-hydroxyindol-3-yl]glyoxylamide semi-ethyl acetate (AWD 12-298) or the pharmacologically acceptable salts thereof.

3. The use as claimed in claim 1 or 2, characterized in that the nonallergic rhinitis is selected from vasomotor rhinitis, nonallergic rhinitis with eosinophils syndrome, chronic sinusitis and rhinitis medicamentosa.

4. The use as claimed in claim 1 or 2, characterized in that the rhinitis caused by infection is a symptom, not caused by allergy, of a viral or bacterial infection or of an infection with fungi or parasites or of a combination of the germs mentioned, selected from vasomotor rhinitis, nonallergic rhinitis with eosinophils syndrome, chronic sinusitis and rhinitis medicamentosa.
